(19) United States
(12) Patent Application Publication
(10) Pub. No.: US 2023/0181624 A1
(43) Pub. Date: Jun. 15, 2023

(54) COMPOSITIONS AND METHODS FOR TREATMENT OF GASTROINTESTINAL BLEEDING

(71) Applicant: Intact Therapeutics, Inc., Belmont, CA (US)

(72) Inventors: Ravinder D. Pamnani, Los Altos, CA (US); Sidhartha Ranjit Sinha, San Francisco, CA (US)

(21) Appl. No.: 17/920,897

(22) PCT Filed: Apr. 20, 2021
(86) PCT No.: PCT/US21/28073

§ 371 Date: Oct. 24, 2022

Related U.S. Application Data
(60) Provisional application No. 63/014,874, filed on Apr. 24, 2020.

(51) Int. Cl. A61K 31/77; A61K 31/195; A61P 7/04; A61K 9/08

(52) U.S. Cl. CPC A61K 31/77; A61K 31/195; A61P 7/04; A61K 9/08

(57) ABSTRACT

The invention provides compositions and methods for treatment of gastrointestinal bleeding, such as upper GI bleeding. In certain aspects, the invention provides topical formulations that stop bleeding in the GI tract by forming a gel when a polymer in the formulation exceeds a threshold level (optionally a pre-determined threshold level) for a property of the polymer of the formulation, which threshold level may be for example, a physical (e.g., temperature), chemical (e.g., pH), or temporal threshold level following contact with the affected tissue. In certain aspects, a combination of properties, such as a combination of physical (e.g., temperature), chemical (e.g., pH), or temporal threshold levels determines when the polymer in the formulation transitions from a liquid to a gel. Methods of using such compositions are also provided.

### CROSS-REFERENCE TO RELATED APPLICATIONS

This application claims the benefit of, and priority to, U.S. Provisional Patent Application No. 63/014,874, filed April 24, 2020, the contents of which are incorporated by reference.

### FIELD OF THE INVENTION

The invention relates generally to compositions and methods for treatment of gastrointestinal bleeding.

### BACKGROUND

Bleeding of the upper gastrointestinal (GI) tract, including the esophagus, stomach, and duodenum, affects about one in 1000 adults each year. Upper GI bleeding may have a variety of causes, such as peptic ulcers, gastric erosions, esophageal varices, and gastric cancer. Severe bleeding of the upper GI tract is a medical emergency; mortality rates are 11% for patients admitted to the hospital because of upper GI bleeding and 33% for patients who develop it while hospitalized.

Existing treatments for treating bleeding of the upper GI tract are inadequate. For example, proton pump inhibitors are often given in the emergent setting, but evidence that such medications decrease death rates, re-bleeding events, or needs for surgical interventions is conflicting. Intravenously administered tranexamic acid (TXA) reduces the risk of death and surgical intervention from upper GI bleeding. However, systemic administration of TXA has serious side effects and is contraindicated for patients at risk of developing thrombosis. Other treatments focus on addressing the underlying cause of bleeding, such as ulcers or varices, or its secondary effects, such as fluid loss or airway obstruction. Nonetheless, the bleeding can still be fatal if the patient does not receive a timely blood transfusion. Consequently, upper GI bleeding remains a serious medical problem, and reliable therapies for treating it are lacking.

### SUMMARY

The invention provides topical formulations that stop bleeding in the GI tract by forming a gel when a polymer in the formulation exceeds a threshold level (optionally a pre-determined threshold level) for a property of the polymer of the formulation, which threshold level may be for example, a physical (e.g., temperature), chemical (e.g., pH), or temporal threshold level following contact with the affected tissue. In certain aspects, a combination of properties, such as a combination of physical (e.g., temperature), chemical (e.g., pH), or temporal threshold levels determines when the polymer in the formulation transitions from a liquid to a gel.

In certain embodiments, the formulations contain polymers that trigger the formulations to undergo a conditional, e.g., temperature-dependent, phase transition. In temperature-dependent formulations, the polymers exist dispersed in a liquid, such as in solution, colloid, suspension, or emulsion, at sub-physiological temperatures, which allows the formulations to be provided to patients in liquid form. At physiological temperatures (i.e., at or near 37° C.), however, the polymers aggregate to form a gel that coats the mucosa of the GI tract and promotes blood clotting. Consequently, the formulations can be delivered topically, e.g., by drinking, via nasogastric tube, or endoscopically, to treat upper GI bleeding. The invention includes methods of treating GI bleeding by topical delivery of such formulations.

The compositions and methods of the invention offer superior efficacy and flexibility in the treatment of upper or lower GI bleeding. Interestingly, the data herein show that the polymers alone in the composition have therapeutic efficacy to stop GI bleeding. That is, the polymers, without any additional antihemorrhagic agents, are the active ingredient in the formulations and act to stop GI bleeding. In other embodiments, for example, the data also show that the formulations may include additional antihemorrhagic agents that facilitate the inherent blood-clotting activity of the aggregated polymers, permitting even quicker cessation of bleeding. In addition, the use of topical formulations avoids the risks associated with systemically administered drugs, so the treatments are safe for patients at risk of developing thrombosis. On the other hand, for patients who are not at such risk, the topical formulations may be combined with systemic therapies to improve the overall efficacy of treatment.

In an aspect, the invention provides topical formulations for treatment of upper gastrointestinal (GI) bleeding in a subject. The formulations include a polymer that exists dispersed in a liquid when the formulation is below a threshold condition and in an aggregated form (e.g., a gel) when the formulation is above the threshold condition, the aggregated form being effective to reduce bleeding in an upper gastrointestinal tract of a subject.

The threshold condition may include one or more of a physical, chemical, and temporal condition. The threshold condition may be any combination of physical, chemical, and temporal conditions.

The physical condition may be temperature. The threshold condition may be a transition temperature. The polymer may exist dispersed in a liquid when the formulation is below the transition temperature and in an aggregated form when the formulation is above the transition temperature. The polymer may exist dispersed in a liquid when the formulation is above the transition temperature and in an aggregated form when the formulation is above the transition temperature.

The chemical condition may be acidity, alkalinity, or pH. The threshold condition may be a transition pH. The polymer may exist dispersed in a liquid when the formulation is below the transition pH and in an aggregated form when the formulation is above the transition pH. The polymer may exist dispersed in a liquid when the formulation is above the transition pH and in an aggregated form when the formulation is above the transition pH.

The temporal condition may be time. The threshold condition may be transition time point. The polymer may exist dispersed in a liquid prior to a transition time point and in an aggregated form after the transition time point. The polymer may exist dispersed in a liquid after a transition time point and in an aggregated form prior to the transition time point.

The formulation may exist as a solution, colloid, suspension, or emulsion when the formulation is below the threshold condition.

The formulation may exist in a liquid phase below the threshold condition and in a gel phase above the threshold condition. The polymer may be a block copolymer. The block copolymer may include one or more of polyethylene glycol and polypropylene glycol.

The formulation may be substantially free of antihemorrhagic agents other than the polymer.

The transition temperature may be from about 4° C. to about 38° C., from about 8° C. to about 38° C., from about 12° C. to about 38° C., from about 16° C. to about 38° C., from about 20° C. to about 38° C., from about 24° C. to about 38° C., from about 28° C. to about 38° C., from about 32° C. to about 38° C., from about 4° C. to about 36° C., from about 8° C. to about 36° C., from about 12° C. to about 36° C., from about 16° C. to about 36° C., from about 20° C. to about 36° C., from about 24° C. to about 36° C., from about 28° C. to about 36° C., from about 32° C. to about 36° C., from about 4° C. to about 34° C., from about 8° C. to about 34° C., from about 12° C. to about 34° C., from about 16° C. to about 34° C., from about 20° C. to about 34° C., from about 24° C. to about 34° C., from about 28° C. to about 34° C., from about 32° C. to about 34° C., from about 4° C. to about 32° C., from about 8° C. to about 32° C., from about 12° C. to about 32° C., from about 16° C. to about 32° C., from about 20° C. to about 32° C., from about 24° C. to about 32° C., from about 28° C. to about 32° C., from about 4° C. to about 30° C., from about 8° C. to about 30° C., from about 12° C. to about 30° C., from about 16° C. to about 30° C., from about 20° C. to about 30° C., from about 24° C. to about 30° C., from about 28° C. to about 30° C., from about 4° C. to about 28° C., from about 8° C. to about 28° C., from about 12° C. to about 28° C., from about 16° C. to about 28° C., from about 20° C. to about 28° C., from about 24° C. to about 28° C., from about 4° C. to about 24° C., from about 8° C. to about 24° C., from about 12° C. to about 24° C., from about 16° C. to about 24° C., from about 20° C. to about 24° C., from about 4° C. to about 20° C., from about 8° C. to about 20° C., from about 12° C. to about 20° C., from about 16° C. to about 20° C., from about 4° C. to about 16° C., from about 8° C. to about 16° C., from about 12° C. to about 16° C., from about 4° C. to about 12° C., or from about 8° C. to about 12° C.

The transition pH may be about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, or about 13.0.

The transition time point may be about 15 seconds, about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, or about 12 minutes, about 15 minutes, about 20 minutes, or about 30 minutes.

The bleeding may be in the upper GI tract. The bleeding may be in the lower GI tract. The bleeding may be in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The bleeding may be associated with one or more conditions. The condition may be caustic ingestion, Dieulafoy's lesions, duodenal ulcer, esophageal cancer, esophageal dysplasia, esophageal ulcers, esophageal varices, esophagitis, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, vascular malformation, polyps, infectious colitis, hemorrhoids, diverticular disease, ischemic colitis, vasculitis, colonic dysplasia, and angiodysplasia.

The formulation may be a consumable beverage. The formulation may be formulated for nasogastric administration. The formulation may be formulated for administration via an endoscope. The formulation may be formulated for rectal administration, for example, including but not limited to by enema, suppository, or via an endoscope. The formulation may include a lipid. The lipid may be a fatty acid, glycolipid, phosphoglyceride, phospholipid, sphingolipid, or sterol. The lipid may be synthetic or naturally-occurring. The lipid may be dipalmitoylphosphatidylcholine (DPPC), di stearoylphosphatidylcholine (DSPC), dimyristoylphosphatidylcholine (DMPC), lecithin, glucosyl-cerebroside, phosphatidylcholine, phosphatidylethanolamine, phosphatidylinositol, phosphatidylserine, or sphingomyelin. The formulation may include liposomes, a lipid coating, or a lipid complex.

In another aspect, the invention provides methods of treating upper or lower gastrointestinal bleeding in a subject. The methods include providing locally to an upper or lower gastrointestinal tract of a subject a formulation containing a polymer that exists dispersed in a liquid when the formulation is below a threshold condition and in an aggregated form when the formulation is above the threshold condition. Providing the formulation may reduce bleeding in the upper or lower gastrointestinal tract of the subject.

The threshold condition may be any of those described above. The threshold condition may be a transition temperature.

The formulation may exist as a solution, colloid, suspension, or emulsion when the formulation is below the threshold condition.

The formulation may be provided in the liquid phase and transition to the gel phase upon exposure to the upper gastrointestinal tract.

The formulation may be provided in the liquid phase and transition to the gel phase upon exposure to the lower gastrointestinal tract.

The formulation may be provided as a consumable beverage. The formulation may be provided by nasogastric administration. The formulation may be administered via an endoscope.

The formulation may exist in a liquid phase below the threshold condition and in a gel phase above the threshold condition.

The polymer may be a block copolymer. The block copolymer may include one or more of polyethylene glycol and polypropylene glycol.

The formulation may be substantially free of antihemorrhagic agents other than the polymer.

The formulation may have any suitable transition temperature, transition pH, or transition time point, such as any of those described above.

The formulation may include a lipid, such as any of those described above. The formulation may include liposomes, a lipid coating, or a lipid complex.

The bleeding may be in the upper GI tract. The bleeding may be in the lower GI tract. The bleeding may be in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The bleeding may be associated with one or more of the conditions described above. In another aspect, the invention provides topical formulations for treatment of upper gastrointestinal bleeding in a subject. The formulations include a polymer that exists dispersed in a liquid when the formulation is below a threshold condition and in an aggregated form when the formulation is above the threshold condition, the aggregated form being effective to reduce bleeding in an upper gastrointestinal tract of a subject and an antihemorrhagic agent that is different from the polymer.

The threshold condition may be any of those described above. The threshold condition may be a transition temperature.

The formulation may exist as a solution, colloid, suspension, or emulsion when the formulation is below the threshold condition.

The formulation may exist in a liquid phase below the threshold condition and in a gel phase above the threshold condition.

The polymer may be a block copolymer. The block copolymer may include one or more of polyethylene glycol and polypropylene glycol.

The formulation may have any suitable transition temperature, transition pH, or transition time point, such as any of those described above.

The bleeding may be in the upper GI tract. The bleeding may be in the lower GI tract. The bleeding may be in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The bleeding may be associated with one or more of the conditions described above.

The antihemorrhagic agent may be aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, or zeolite.

The formulation may be a consumable beverage. The formulation may be formulated for nasogastric administration. The formulation may be formulated for administration via an endoscope.

The formulation may include a lipid, such as any of those described above. The formulation may include liposomes, a lipid coating, or a lipid complex.

In another aspect, the invention provides methods of treating gastrointestinal bleeding in a subject. The methods include providing locally to an upper or lower gastrointestinal tract of a subject a formulation containing (1) a polymer that exists dispersed in a liquid when the formulation is below a threshold condition and in an aggregated form when the formulation is above the threshold condition and (2) an antihemorrhagic agent that is different from the polymer. Providing the formulation may reduce bleeding in the upper or lower gastrointestinal tract of the subject.

The threshold condition may be any of those described above. The threshold condition may be a transition temperature.

The formulation may exist as a solution, colloid, suspension, or emulsion when the formulation is below the threshold condition.

The formulation may exist in a liquid phase below the threshold condition and in a gel phase above the threshold condition.

The formulation may be provided in the liquid phase and transition to the gel phase upon exposure to the upper or lower gastrointestinal tract.

The polymer may be a block copolymer. The block copolymer may include one or more of polyethylene glycol and polypropylene glycol. The formulation may have any suitable transition temperature, transition pH, or transition time point, such as any of those described above.

The bleeding may be in the upper GI tract. The bleeding may be in the lower GI tract. The bleeding may be in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The bleeding may be associated with one or more of the conditions described above.

The antihemorrhagic agent may be desmopressin, aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, or zeolite.

The formulation may be provided as a consumable beverage. The formulation may be provided by nasogastric administration. The formulation may be administered via an endoscope.

The formulation may include a lipid, such as any of those described above. The formulation may include liposomes, a lipid coating, or a lipid complex.

In another aspect, the invention provides methods of preventing a recurrence of gastrointestinal bleeding in a subject that has previously had gastrointestinal bleeding. The methods include providing locally to a gastrointestinal tract of a subject that has previously had gastrointestinal bleeding a formulation containing a polymer that exists dispersed in a liquid when the formulation is below a threshold condition and in an aggregated form when the formulation is above the threshold condition. Providing the formulation may prevent a recurrence of bleeding in the gastrointestinal tract.

The threshold condition may be any of those described above. The threshold condition may be a transition temperature.

The formulation may exist as a solution, colloid, suspension, or emulsion when the formulation is below the threshold condition.

The formulation may be provided in the liquid phase and transition to the gel phase upon exposure to the upper gastrointestinal tract.

The formulation may be provided in the liquid phase and transition to the gel phase upon exposure to the lower gastrointestinal tract.

The formulation may be provided as a consumable beverage. The formulation may be provided by nasogastric administration. The formulation may be administered via an endoscope.

The formulation may exist in a liquid phase below the threshold condition and in a gel phase above the threshold condition.

The polymer may be a block copolymer. The block copolymer may include one or more of polyethylene glycol and polypropylene glycol.

The formulation may be substantially free of antihemorrhagic agents other than the polymer.

The formulation may have any suitable transition temperature, transition pH, or transition time point, such as any of those described above.

The formulation may include a lipid, such as any of those described above. The formulation may include liposomes, a lipid coating, or a lipid complex.

The previous bleeding may have been in the upper GI tract. The previous bleeding may have been in the lower GI tract. The previous bleeding may have been in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The bleeding may be associated with one or more of the conditions described above.

In another aspect, the invention provides methods of preventing a recurrence of gastrointestinal bleeding in a subject that has previously had gastrointestinal bleeding. The methods include providing locally to an upper or lower gastrointestinal tract of a subject a formulation containing (1) a polymer that exists dispersed in a liquid when the formulation is below a threshold condition and in an aggregated form when the formulation is above the threshold condition and (2) an antihemorrhagic agent that is different from the polymer. Providing the formulation may prevent a recurrence of bleeding in the gastrointestinal tract.

The threshold condition may be any of those described above. The threshold condition may be a transition temperature.

The formulation may exist as a solution, colloid, suspension, or emulsion when the formulation is below the threshold condition.

The formulation may exist in a liquid phase below the threshold condition and in a gel phase above the threshold condition.

The formulation may be provided in the liquid phase and transition to the gel phase upon exposure to the upper or lower gastrointestinal tract.

The polymer may be a block copolymer. The block copolymer may include one or more of polyethylene glycol and polypropylene glycol.

The formulation may have any suitable transition temperature, transition pH, or transition time point, such as any of those described above.

The previous bleeding may have been in the upper GI tract. The previous bleeding may have been in the lower GI tract. The previous bleeding may have been in one or more of the mouth, pharynx, esophagus, stomach, small intestine, duodenum, jejunum, ileum, large intestine, cecum, colon, ascending colon, transverse colon, descending colon, sigmoid colon, rectum, and anus.

The previous bleeding may be associated with one or more of the conditions described above.

The antihemorrhagic agent may be desmopressin, aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, or zeolite.

The formulation may be provided as a consumable beverage. The formulation may be provided by nasogastric administration. The formulation may be administered via an endoscope.

The formulation may include a lipid, such as any of those described above. The formulation may include liposomes, a lipid coating, or a lipid complex.

### BRIEF DESCRIPTION OF THE DRAWINGS

FIG. 1 is a schematic illustrating the mechanism for treatment of upper GI bleeding according to embodiments of the invention.

FIG. 2 is a schematic of the phase transition of formulations in embodiments of the invention.

FIG. 3 is a graph of the relationship between storage modulus and gel transition temperature for formulations containing Poloxamer 407. Inset are images of tubes containing compositions at 27° C. (light blue arrow) and 39° C. (dark blue arrow) from data points on the graph.

FIG. 4 shows CT images of mice following rectal administration of water or thermogel compositions.

FIG. 5 is a graph of volume retained as a function of time following rectal administration of water or thermogel compositions.

FIG. 6 is an image of an actively bleeding tail in a mouse tail-snip assay.

FIG. 7 is an image of a tail in which bleeding has ceased in a mouse tail-snip assay.

FIG. 8 is a graph showing bleeding time of mouse tails exposed to various formulations in a mouse tail-snip assay.

### DETAILED DESCRIPTION

The invention provides compositions and methods for treating bleeding of the gastrointestinal (GI) tract or regions of the GI tract. The compositions include topical formulations containing polymers that trigger the formulations to undergo a conditional transition. For example, the formulations may transition from a liquid to a gel when a polymer in the formulation exceeds a threshold level (optionally a pre-determined threshold level) for a property of the polymer of the formulation, which threshold level may be for example, a physical (e.g., temperature), chemical (e.g., pH), or temporal threshold level following contact with the affected tissue. In certain aspects, a combination of properties, such as a combination of physical (e.g., temperature), chemical (e.g., pH), or temporal threshold levels determines when the polymer in the formulation transitions from a liquid to a gel.

In temperature-dependent embodiments of the formulations, the polymers exist dispersed in a liquid at sub-physiological temperatures but aggregate at physiological temperatures (i.e., at or near 37° C.) to form a gel that promotes blood clotting. Because the temperature-dependent formulations are liquids at lower temperatures, they are easy to administer to patients and deliver to affected tissues. Moreover, by acting topically, the formulations avoid the systemic side effects associated with drugs given intravenously. In certain embodiments, the polymers themselves are the active agent that acts to stop the bleeding, i.e., the formulations do not include any other agent or agents that have antihemorrhagic properties. Rather, the polymers are provided in a therapeutic amount that upon transition from a liquid to a gel, the gelled polymers stop GI bleeding. In other embodiments of the invention, the formulations also contain one or more antihemorrhagic agents that act synergistically with the aggregated polymer to reduce bleeding. The invention also provides methods of treating upper or lower GI bleeding using the formulations described herein.

### Compositions

The invention provides topical formulations that contain one or more polymers that induce a conditional phase transition in the formulation. The polymer exists dispersed in a liquid when the formulation is below a threshold condition, e.g., a transition temperature, and forms aggregates that cause the formulation to gel above the threshold condition. The present invention recognizes that the polymer aggregates in such formulations have inherent antihemorrhagic activity. Consequently, topical formulations containing such polymers are useful to reduce or prevent bleeding in the upper or lower GI tract, and the clot-promoting activity is increased in formulations that also contain additional antihemorrhagic agents.

The threshold condition may include one or more of a physical, chemical, and temporal condition. The threshold condition may be any combination of physical, chemical, and temporal conditions.

The physical condition may be temperature. The threshold condition may be a transition temperature. The polymer may exist dispersed in a liquid when the formulation is below the transition temperature and in an aggregated form when the formulation is above the transition temperature. The polymer may exist dispersed in a liquid when the formulation is above the transition temperature and in an aggregated form when the formulation is above the transition temperature.

The chemical condition may be acidity, alkalinity, or pH. The threshold condition may be a transition pH. The polymer may exist dispersed in a liquid when the formulation is below the transition pH and in an aggregated form when the formulation is above the transition pH.

The polymer may exist dispersed in a liquid when the formulation is above the transition pH and in an aggregated form when the formulation is above the transition pH.

The temporal condition may be time. The threshold condition may be transition time point. The polymer may exist dispersed in a liquid prior to a transition time point and in an aggregated form after the transition time point. The polymer may exist dispersed in a liquid after a transition time point and in an aggregated form prior to the transition time point.

In the liquid phase, the formulation may exist as a solution, colloid, suspension, or emulsion when the formulation is below the transition temperature. The bulk phase of the liquid may be an aqueous medium. In some embodiments, the polymers are water-soluble and dissolve in the aqueous medium. In some embodiments, the polymers are insoluble or immiscible in water and form colloids, suspensions, or emulsions. The formulations may be homogeneous or heterogeneous in the liquid phase.

The formulations may contain one or more polymers that promote a conditional, e.g., temperature-dependent, transition from a liquid phase to a gel phase. The polymer may be a block copolymer. The block polymer may include blocks of a relatively hydrophilic polymer, such as polyethylene glycol, and blocks of a relatively hydrophobic polymer, such as polypropylene glycol. The polymer may be a natural polymer. For example and without limitation, the natural polymer may be pectin, xyloglucan, gellan gum, chitosan, or alginic acid. The polymer may be an inorganic polymer. For example and without limitation, the polymer may be or contain silicon oxide. Topical formulations containing polymers that promote temperature-dependent phase transitions are known in the art and described in, for example, International Patent Publication No. WO 2016/179227; and Sidhartha R. Sinha, et al., A Thermo-Sensitive Delivery Platform for Topical Administration of Inflammatory Bowel Disease Therapies, Gastroenterology, 2015 July; 149(1):52-55.e2, doi: 10.1053/j.gastro.2015.04.002, the contents of each of which are incorporated herein by reference.

The formulation may contain the polymer at a defined concentration. For example and without limitation, the formulation may contain the polymer at about 10%, about 12%, about 14%, about 16%, about 17%, about 17.5%, about 18%, about 18.5%, about 19%, about 19.5%, about 20%, about 20.5%, about 21%, about 22%, about 24%, or about 26% by weight. Formulations of the invention exist in liquid form below a transition temperature and in gel form above a transition temperature. The transition temperature may be at or near the physiological temperature of a human, or it may be below the physiological temperature of a human. For example and without limitation, the transition temperature may be from about 4° C. to about 38° C., from about 8° C. to about 38° C., from about 12° C. to about 38° C., from about 16° C. to about 38° C., from about 20° C. to about 38° C., from about 24° C. to about 38° C., from about 28° C. to about 38° C., from about 32° C. to about 38° C., from about 4° C. to about 36° C., from about 8° C. to about 36° C., from about 12° C. to about 36° C., from about 16° C. to about 36° C., from about 20° C. to about 36° C., from about 24° C. to about 36° C., from about 28° C. to about 36° C., from about 32° C. to about 36° C., from about 4° C. to about 34° C., from about 8° C. to about 34° C., from about 12° C. to about 34° C., from about 16° C. to about 34° C., from about 20° C. to about 34° C., from about 24° C. to about 34° C., from about 28° C. to about 34° C., from about 32° C. to about 34° C., from about 4° C. to about 32° C., from about 8° C. to about 32° C., from about 12° C. to about 32° C., from about 16° C. to about 32° C., from about 20° C. to about 32° C., from about 24° C. to about 32° C., from about 28° C. to about 32° C., from about 4° C. to about 30° C., from about 8° C. to about 30° C., from about 12° C. to about 30° C., from about 16° C. to about 30° C., from about 20° C. to about 30° C., from about 24° C. to about 30° C., from about 28° C. to about 30° C., from about 4° C. to about 28° C., from about 8° C. to about 28° C., from about 12° C. to about 28° C., from about 16° C. to about 28° C., from about 20° C. to about 28° C., from about 24° C. to about 28° C., from about 4° C. to about 24° C., from about 8° C. to about 24° C., from about 12° C. to about 24° C., from about 16° C. to about 24° C., from about 20° C. to about 24° C., from about 4° C. to about 20° C., from about 8° C. to about 20° C., from about 12° C. to about 20° C., from about 16° C. to about 20° C., from about 4° C. to about 16° C., from about 8° C. to about 16° C., from about 12° C. to about 16° C., from about 4° C. to about 12° C., or from about 8° C. to about 12° C.

The transition from liquid to gel may occur above or below a transition pH. The transition pH may be about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5, about 12.0, about 12.5, or about 13.0.

The transition from liquid to gel may occur after a transition time. The transition time point may be about 15 seconds, about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, or about 12 minutes, about 15 minutes, about 20 minutes, or about 30 minutes.

The formulation may contain a lipid. The lipid may be a fatty acid, glycolipid, phosphoglyceride, phospholipid, sphingolipid, or sterol. The lipid may be synthetic or naturally-occurring. For example and without limitation, the lipid may be dipalmitoylphosphatidylcholine (DPPC), distearoylphosphatidylcholine (DSPC), glucosyl-cerebroside, phosphatidylcholine, phosphatidylethanolamine, phosphatidylinositol, phosphatidylserine, or sphingomyelin.

The formulation may include one or more types of lipid-based structures. For example and without limitation, the formulation may include liposomes, a lipid coating, or a lipid complex.

The formulation may contain one or more excipients. The excipient may promote the mucoadhesion properties of the gelled form. For example and without limitation, the excipient may be poly(acrylic acid), poly-vinyl-alcohol, sodium-carboxy-methyl-cellulose, or sodium alginate. Other excipients may include pH adjusting agents, antioxidants, solubilisers, and preservatives.

The formulation may contain one or more molecules that exist as free molecules dispersed in a liquid, e.g., in solution, colloid, suspension, or emulsion, but form polymers in response to a stimulus, thereby promoting transition to a gel phase. Such molecules may be organic molecules, inorganic molecules, or macromolecule, e.g., proteins. For example, under acidic conditions certain mucins undergo a conformational change that allows them to polymerize. Consequently, reducing the pH of concentrated mucin solutions can trigger gel formation. In another example, mixtures of polyacrylic acid and guanidium exist as liquid solutions at pH of <4 but transition to gels at neutral pH due to the formation of a supramolecular poly-electrolyte complex (SPEC). For example and without limitation, the stimulus that promotes polymerization and/or gel formation may be a divalent cation, e.g., calcium or magnesium, an epoxy, an acid, or a base.

The formulation may be substantially free of antihemorrhagic agents other than the one or more polymers that promote the temperature-dependent phase transition of the formulation. Alternatively, the formulation may contain one or more antihemorrhagic agents in addition to the one or more polymers that promote the phase transition. For example and without limitation, the additional antihemorrhagic agent may be aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, or zeolite.

The formulation may contain one or more additional therapeutic agents that are not antihemorrhagic or that are included for a purpose other than to stop bleeding. For example and without limitation, the formulation may contain an antibiotic, anti-inflammatory, prokinetic, antacid, or proton pump inhibitor. For example and without limitation, the formulation may contain one or more of dexlansoprazole, esomeprazole, lansoprazole, omeprazole, pantoprazole, rabeprazole, and sodium bicarbonate.

The formulation may contain an antihemorrhagic agent in a complex with, or conjugated to, a polymer. Without wishing to be bound by theory, the polymer may form micelles that retain the agent in the formulation in the liquid phase. At higher temperatures, dehydration of the micelles may cause them to aggregate to form a gel while releasing the agent so that it can enter the tissue of the colon. The formulation may contain lipid that encapsulates the antihemorrhagic agent or forms a complex with antihemorrhagic agents. Alternatively or additionally, the formulation may contain a free lipid dissolved or emulsified in the formulation.

The formulation may be designed for a particular route and/or mode of administration. For example, the formulation may be, or may be a component of, a consumable beverage that may be drunk by the subject. Alternatively or additionally, the formulation may be suitable for administration bronchoscopically, by catheter (including cardiac catheter), endoscopically, by enema, by injection, or nasogastrically.

### Methods

The invention provides methods of treating or preventing upper or lower GI bleeding in a subject by providing locally a formulation that contains one or more polymers that induce a conditional transition in the formulation from a liquid to a gel. The formulation may be provided in the liquid phase and transition into the gel phase upon exposure to the GI tract. In certain embodiments, the formulation transitions from a liquid to a gel when a polymer in the formulation exceeds a threshold level (optionally a pre-determined threshold level) for a property of the polymer of the formulation, which threshold level may be for example, a physical (e.g., temperature), chemical (e.g., pH), or temporal threshold level following contact with the affected tissue. In certain aspects, a combination of properties, such as a combination of physical (e.g., temperature), chemical (e.g., pH), or temporal threshold levels determines when the polymer in the formulation transitions from a liquid to a gel. In certain embodiments, the formulation may be provided at a temperature below a threshold condition, e.g., a transition temperature, and increase to above the threshold condition. The transition may occur in the body of the subject.

The method may stop, reduce, or prevent upper or lower GI bleeding. Reduction of bleeding may be assessed by any suitable measure. For example and without limitation, reduction of bleeding may be assessed by a change in one or more of the following: time until bleeding stops, volume of blood loss, volume of blood transfusion, blood cell count, non-cellular blood markers (e.g., hemoglobin, blood urea nitrogen), blood pressure, pulse, volume of vomiting, frequency of vomiting, consistency of vomit, hematochezia, stool consistency, and stool color. The method may reduce bleeding, as assessed by any of the aforementioned measures, by a defined amount. For example and without limitation, the method may reduce bleeding by about 10%, about 20%, about 30%, about 40%, about 50%, about 60%, about 70%, about 80%, about 90%, or about 100%.

The method may reduce or prevent future occurrences of bleeding. Any of the aforementioned criteria may be used to assess reduction of future occurrences of bleeding. Future occurrences of bleeding may be reduced by a defined amount, such as any of those described above. Prevention of bleeding may be defined as the absence of bleeding over a period of time. For example and without limitation, prevention may be an absence of bleeding for 1 hour, 2 hours, 4 hours, 6 hours, 8 hours, 12 hours, 1 day, 2 days, 3 days, 4 days, 5 days, 1 week, 2 weeks, 4 weeks, 6 weeks, 8 weeks, 12 weeks, or more.

The formulation may be provided to the subject by any suitable manner. For example, the formulation may be provided as, or contained within a beverage. The formulation may be administered nasogastrically. The formulation may be administered via bronchoscope, catheter, endoscope, enema, or injection.

The formulation may be provided at a temperature at or near its transition temperature. The formulation may be provided below its transition temperature. Prior to administering the formulation to the subject, the formulation may be stored at room temperature or with refrigeration.

The formulation may be provided at a pH at or near its transition pH. The formulation may be provided below or above its transition pH.

The method may be performed without the use of a systemically administered antihemorrhagic agent. Antihemorrhagic agents that enter the blood increase the risk that a subject will develop a thrombus, and circulating blood clots can lead to strokes, heart attacks, and pulmonary embolisms. Therefore, local delivery of a formulation of the invention without the use of a systemic antihemorrhagic agent may be useful to treat patients for whom blood clots present a serious health risk, such as patients having one or more of the following conditions: aneurysm, angina, aortic aneurysm, atherosclerosis, atrial fibrillation, blood clots, cardiac dysrhythmias, cancer, cardiomyopathy, carotid artery disease, cerebrovascular disease, congenital heart disease, coronary artery disease, deep vein thrombosis, endocarditis, eosinophilic myocarditis, heart attack, heart failure, heart valve replacement, hypertension, hypertensive heart disease, inflammatory cardiomegaly, inflammatory heart disease, myeloproliferative disorders, myocarditis, peripheral arterial disease, prothrombotic coagulation abnormalities, pulmonary embolism, pregnancy, pulmonary heart disease, Raynaud's disease, renal artery stenosis, restenosis, rheumatic heart disease, sepsis, stroke, valvular heart disease, varicose veins, and vasculitis. Local delivery of a formulation of the invention without the use of a systemic antihemorrhagic agent may also be useful to treat patients who take anticoagulants, such as antithrombin, apixaban, argatroban, batroxobin, betrixaban, bivalirudin, coumarin, dabigatran, darexaban, a direct factor Xa inhibitor, a direct thrombin inhibitor, edoxaban, eribaxaban, fondaparinux, hementin, heparin, hirudin, idrabiotaparinux, idraparinux, lepirudin, letaxaban, rivaroxaban, vitamin E, warfarin, and ximelagatran, or patients who take agglutination inhibtors, such as acetylsalicylic acid, clopidogrel, prasuragel, and ticagrelor.

Alternatively, the method may include providing formulation of the invention locally in combination with systemic administration of one or more other antihemorrhagic agents, such as any of those described above. Such combination therapies may be suitable for patients that do not have, or are not at risk of developing, a condition in which blood clots present a serious health risk, such as any of those described above, or for patients who do not take an anticoagulant or a platelet agglutination inhibitor, such as one of those described above.

The systemic antihemorrhagic agent may be provided by any suitable route. For example and without limitation, the systemic antihemorrhagic agent may be provided intravenously, intraarterially, by inhalation, orally, enterally, parenterally, subcutaneously, by injection, or by infusion.

The subject may be an animal. For example, the subject may be a mammal, such as a human. The subject may be a pediatric, a newborn, a neonate, an infant, a child, an adolescent, a pre-teen, a teenager, an adult, or an elderly subject. The subject may be a member of a population that has or is at risk of developing a condition, such as any of the aforementioned conditions in which blood clots present a serious health risk. The subject may be a member of a population that does not have or is not at high risk of developing a condition, such as any of the aforementioned conditions in which blood clots present a serious health risk. The subject may be a patient who takes or a patient who does not take an anticoagulant, such as any of those described above.

Upper GI bleeding and Other Indications

The compositions and methods of the invention are useful for treating GI bleeding. The bleeding may be in a region of the GI tract. The bleeding may be in the upper GI tract, which includes the mouth, pharynx, esophagus, stomach, and duodenum and/or small intestine, or the lower GI tract, which includes the large intestine. For example and without limitation, the bleeding may be in one or more of the mouth, pharynx, esophagus, stomach, small intestine, duodenum, jejunum, ileum, large intestine, cecum, colon, ascending colon, transverse colon, descending colon, sigmoid colon, rectum, and anus.

Upper GI bleeding may be caused by or associated with another condition. For example, upper GI bleeding may be caused by or associated with caustic ingestion, Dieulafoy's lesions, duodenal ulcer, esophageal cancer, esophageal dysplasia, esophageal ulcers, esophageal varices, esophagitis, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

Upper GI bleeding may result from the use of drugs, such as nonsteroidal anti-inflammatory drugs (NSAIDs), selective serotonin reuptake inhibitors (SSRIs), anticoagulation drugs, and anti-platelet drugs. Upper GI bleeding may be associated with use of one or more drugs, such as aceclofenac, alaproclate, aspirin, celecoxib, centpropazine, cericlamine, citalopram, clonixin, dapoxetine, desvenlafaxine, dexibuprofen, dexketoprofen, diclofenac, diflunisal, droxicam, duloxetine, escitalopram, etodolac, etoricoxib, femoxetine, firocoxib, flufenamic acid, fluoxetine, flurbiprofen, fluvoxamine, genoprofen, H-harpagide, ibuprofen, ifoxetine, indalpine, Indomethacin, isoxicam, ketoprofen, ketorolac, levomilnacipran, licofelone, litoxetine, lornoxicam, loxoprofen, lubazodone, lumiracoxib, meclofenamic acid, mefenamic acid, meloxicam, milnacipran, nabumetone, naproxen, nimesulide, omiloxetine, oxaprozin, panuramine, parecoxib, paroxetine, phenylbutazone, pirandamine, piroxicam, rofecoxib, salicylic acid, salsalate, seproxetine, sertraline, sulindac, tenoxicam, tolfenamic acid, tolmetin, valdecoxib, venlafaxine, vilazodone, vortioxetine, or zimelidine.

The compositions and methods of the invention are useful for treatment of bleeding in other parts of the body as well. In particular, the compositions and methods are useful for treating bleeding of internal tissues and/or of tissues that are difficult to reach. The bleeding may be within an orifice, or it may be internal. For example and without limitation, the bleeding may be in one or more of the abdominal cavity (e.g., the inguinal region), bladder, ear (otorrhagia), eye (e.g., vitreous hemorrhage), lower GI tract, lungs (e.g., bronchial bleeding), nasal cavity (e.g., epistaxis), oral cavity, respiratory tract, tonsils, urinary tract, uterus, vagina, chest cavity, muscle, soft tissue, or pelvic cavity. The bleeding may be caused by, or associated with, endoscopic intervention (e.g., gastric polypectomy), stroke (e.g., hemorrhagic stroke), non-surgical intervention (e.g., polypectomy, tooth extraction, etc.), pregnancy or childbirth (e.g., postpartum bleeding), pseudoaneurysm (e.g., pseudoaneurysm that developed post catheterization), surgery (i.e., postoperative bleeding), tonsillectomy, trauma (e.g., blunt force, projectile, puncture, etc.), or a tumor, including tumors for which no specific lesion is being treated.

### EXAMPLES

The invention provides a thermogel-based platform for the delivery of drugs to stop GI bleeding, including upper GI bleeding (UGIB). The compositions have an innovative formulation designed to provide superior muco-adhesion for the GI tract. The compositions are liquid at ambient temperature and become a gel when heated to body temperature. Their action is based on two synergistic effects. First, the in situ gelation of the muco-adhesive thermogel provides a mechanical barrier against the blood flow. Second, the slow release of drugs loaded in the thermogel enables healing of the hemorrhage site. The compositions are completely safe to use, and they are physiologically expelled from the body with stools after their action is completed. They can be used for the initial stabilization of patients with UGIB. They can be used as a rescue treatment after failure of endoscopic treatment. They can be used as treatment to prevent recurrence of GI bleeding in patients following a previous episode of GI bleeding, either for self-administration at home or administration by a healthcare provider. They represent a disruptive novel solution for the treatment of UGIB in emergency scenarios resulting in a reduced social and financial impact of UGIB on society. The thermogels can be used to treat a variety of other bleeding applications, including trauma, other orifices (e.g., epistaxis, otorrhagia, oral bleeding), or difficult to reach bleeding sites (e.g., internal bleedings). Additionally, the thermogels may be used as a platform in other applications requiring drug delivery to the upper GI tract, such as GERD and eosinophilic esophagitis.

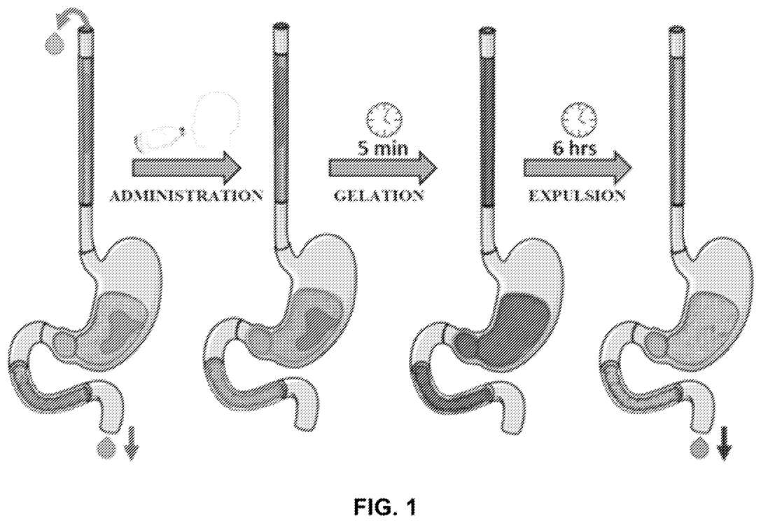

FIG. 1 is a schematic illustrating the mechanism for treatment of upper GI bleeding according to embodiments of the invention.

The aforementioned formulations are liquid at room temperature to allow fast and easy administration as a drinkable liquid or through standard nasogastric (NG) tubes. At body temperature, the formulations quickly become a gel (<1 minute) covering the whole upper GI tract, including esophagus, stomach, and duodenum. Certain embodiments have a custom excipient formulation that ensures unmatched mucoadhesion achieving long retention times (>3 hours). The gel's inherent micellization properties have been designed to effectively stop bleeding. In addition, the formulations can be tuned as a drug carrier to allow topical delivery of the most effective clotting promoters to stop UGIB. The extensive coating of the mucosal wall of the GI tract will allow improved bioavailability and better drug absorption. Such compositions will control the delivery and release of drugs at the site of injury, improving healing rate and decreasing the chances of re-bleeding. The gel is safe to use, and after completing its action (˜1.5 hours), it detaches from the mucosa thanks to natural gastrointestinal movements. The product is later expelled with the stool.

The formulations described herein enable the development of novel products posed to disrupt the pre-endoscopic UGIB management. No product with the same properties or functions currently exists. Prior therapies for treatment of UGIB are described in Table 1.

TABLE 1

| Therapeutic | Hemostatic | | Side- | Time to | |
|---|---|---|---|---|---|
| class | Action | Delivery | effects | hemostasis | |
| Hemostatic | Mechanical | Endoscopic | Mild | <1 | min |
| powders | or | | | | |
| | Biological | | | | |
| Antibiotics | None | Intravenous | Mild | — | |
| & Pro- | | | | | |
| kinetics | | | | | |
| PPIs | Chemical | Intravenous | Moderate | >6 | hours |
| Clotting | Biological | Intravenous | Severe | >30 | min |
| promoters | | | | | |
| Compositions | Mechanical | Topical | None | ~5 | min |
| of the | & | (drink/NG | | | |
| invention | Biological | tube) | | | |

Hemostatic powders are substances that control the active bleeding by forming a solid matrix with a tamponade function over the bleeding site and by boosting platelet aggregation and coagulation cascade. Currently, they are only delivered endoscopically. The pre-endoscopic intravenous (IV) pharmacological treatment of UGIB is controversial. It is common practice to initiate IV proton pump inhibitors (PPIs) therapy once any necessary resuscitative measures have been implemented. PPIs achieve a mild hemostatic effect by neutralizing gastric secretions, thus resulting in a stabilization of blood clots. Nevertheless, it was recently demonstrated that PPIs do not significantly improve any clinically important outcomes such as mortality, re-bleeding, or the requirement for surgery. Additional drugs are usually administered IV, including erythromycin to empty the gastric content and antibiotics to prevent bacterial infections. These drugs do not have direct hemostatic effects but avoid the development of complications. Recently, the IV administration of clotting promoters (e.g. TXA) has been proposed to stabilize UGIB patients' conditions. While a reduced risk of death and need for emergency surgical intervention was observed, the IV administration required high doses and led to systemic side-effects. The compositions of the invention deliver clotting promoter drugs directly at the site of injury, reducing time to hemostasis and with no systemic side-effects.

In about 75% of all GI bleeding cases, the bleeding site is located above the ligament of Treitz (i.e. in the esophagus, stomach or duodenum), resulting in an upper GI bleeding (UGIB), with symptoms including hematemesis (red blood or coffee-ground emesis), melena (black, tarry stool) and hematochezia (red or maroon blood in the stool). Intravenous (IV) drugs are not effective at achieving UGIB hemostasis and can cause systemic side-effects. Topical hemostatic agents are commonly used to control severe bleeding, as they act by adhering to damaged tissues, sealing injured blood vessels, and accelerating the clotting cascade. However, in the case of UGIB, a topical hemostatic has not been used without endoscopy due to the difficulty of delivering them to the bleeding site.

Examples of compositions of the invention and their use are provided in the following examples. The examples are for illustrative purposes only and do not limit the scope of the invention.

### Example 1

Poloxamer is a class of ABA type triblock polymers formed by a central hydrophobic chain of polyoxypropylene (PO) and two lateral hydrophilic chains of polyoxyethylene (EO). Poloxamers are useful in mucosal drug delivery systems, due to their non-irritating action on the mucosa, and to their ability to deliver drugs to a specific compartment, maintaining the required concentration for a prolonged period of time, leading to a decrease in the effective dosage and side effects compared to systemic treatments. Aqueous solutions of poloxamers are liquid at low temperature and form a gel at higher temperatures in a reversible process.

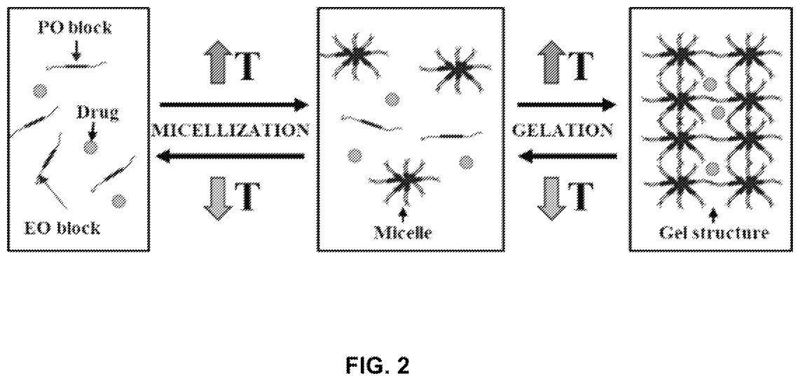

FIG. 2 is a schematic of the phase transition of formulations in embodiments of the invention. At low temperature individual block copolymers are present in solution. By increasing the temperature, micellization (i.e. aggregation of individual blocks) occurs to minimize the interactions of the hydrophobic PO blocks with the solvent. By further increasing the temperature, micelles crystallize into a 3D structure forming a gel. Drug molecules present in solutions are trapped into the solidified gel. Gelation is reversible.

The transition temperature depends on the polymer concentration. Drug molecules loaded in the solutions remain trapped inside the gel, and are released over time by diffusing through the gel. By controlling gel viscosity and the concentration of salts and organic solvents, gelation, mucoadhesive and drug release properties (i.e. diffusion coefficient) are controlled to achieve optimal drug delivery to the GI tract.

### Example 2

The properties of thermogels based on Poloxamer 407 were investigated. Poloxamer 407 has a molecular weight of 12,000 Da and a PEO/PPO ratio of 2:1 by weight, and it is Generally Recognized As Safe (GRAS) by the FDA. Gels of the invention are designed to have superior mucoadhesion properties thanks to the addition of excipients, such as poly(acrylic acid), poly-vinyl-alcohol, sodium-carboxy-methyl-cellulose, sodium alginate, which have charged and uncharged groups able to form electrostatic and hydrogen bonds with the mucosa. This mucoadhesion enables the gels to be retained in the gastrointestinal tract for >3 hours, ensuring sustained drug release. Drugs are released from the solidified Poloxamer 407 thermogel following the Higuchi square root law, with a diffusion coefficient for a medium-sized molecule (lidocaine, ˜234 Da) in the 1 3×10 6 cm²/s range, resulting in ˜60% of the drug being delivered within 3 hours.

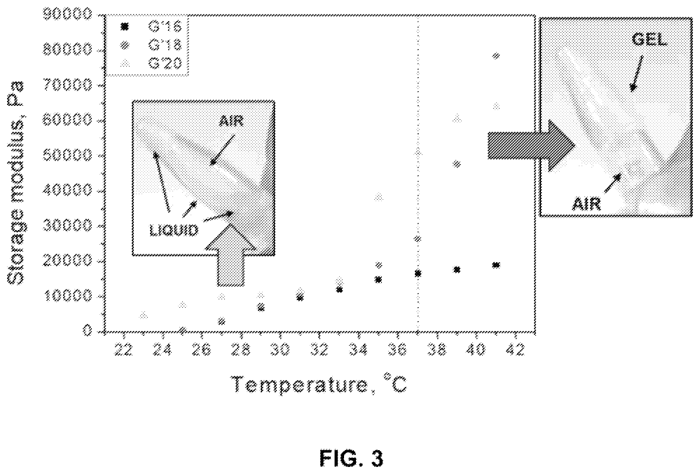

FIG. 3 is a graph of the relationship between storage modulus and gel transition temperature for formulations containing Poloxamer 407. Inset are images of tubes containing compositions at 27° C. (light blue arrow) and 39° C. (dark blue arrow) from data points on the graph. The poloxamer concentration necessary to achieve a transition temperature of ˜36° C. was experimentally determined. The storage modulus of 3 aqueous solutions with different poloxamer concentrations (16%, 18%, 20% w/w) was measured in a rheometer over the temperature range 23-41° C. The transition temperature, defined as the lowest temperature at which the storage modulus is >45kPa, was 39° C. for the 18% solution, 36.5° C. for the 20% solution, and was not in the measured range for the 16% solution. Thus, a poloxamer concentration of 20% has a transition temperature well suited for biological applications and was previously used for the treatment of ulcerative colitis.

### Example 3

The ability of the thermogel to adhere to the gastrointestinal mucosa was tested in a mouse model. Enemas of either liquid water or poloxamer thermogel were administered rectally to mice. The amount of volume retained over time (after 0.5, 1.5 and 3 hours) was measured with Computed Tomography (CT) Imaging.

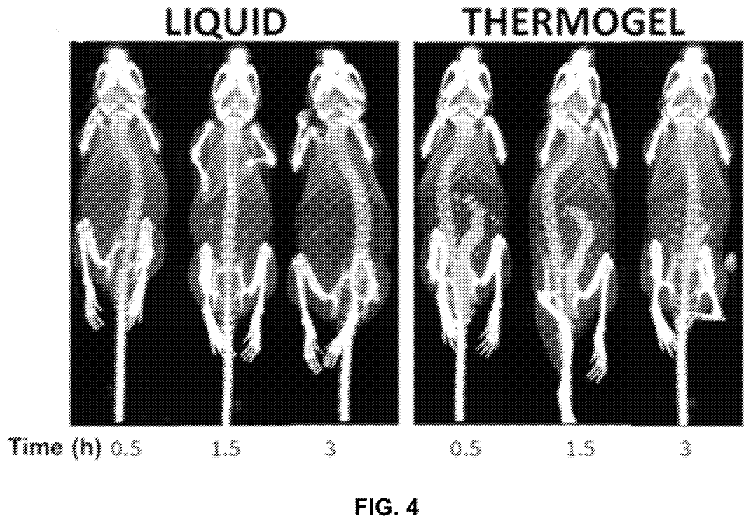

FIG. 4 shows CT images of mice following rectal administration of water or thermogel compositions. Mice in left panel were given liquid water containing a contrast agent, and mice in the right panel were given thermogel containing contrast agent. Images were taken 0.5 hours, 1.5 hours, or 3 hours after administration, as indicated.

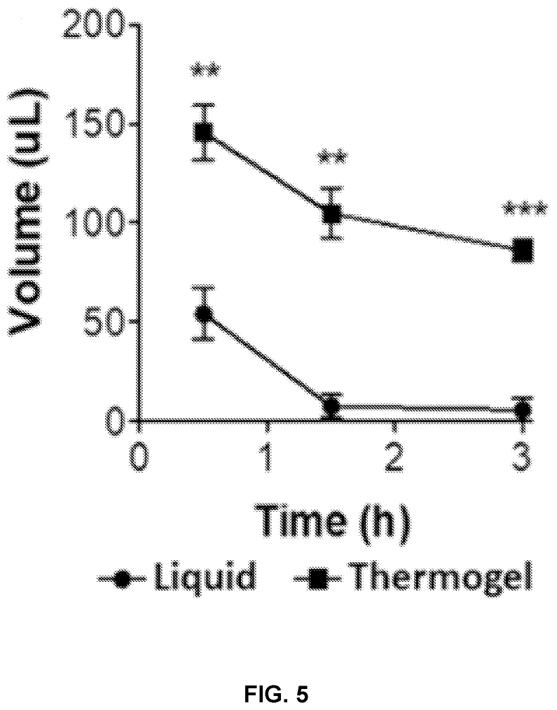

FIG. 5 is a graph of volume retained as a function of time following rectal administration of water or thermogel compositions. Circles represent data from mice given liquid water containing a contrast agent, and square represent data from mice given thermogel containing contrast agent.

The results show that the thermogel was able to stick to the mucosa, with —60% of the volume being retained after 3 hours, significantly better than the liquid enema which was almost entirely expelled (p<0.001). Additionally, after 3 hours the thermogel was still lining the internal surface of the gastrointestinal tract in a continuous coating, showing excellent muco-adhesive properties. The mucoadhesion is affected by the physiological pH which is ˜6.7 in all the lower GI tract, but varies significantly in the upper GI tract (from ˜2.5 in the stomach to ˜7 in the esophagus).

Formulations are modified to achieve the same level of retention properties in the upper GI tract. Compared to the lower GI tract, the upper GI tract has a more acidic environment, and patients have less control over esophageal and gastric movements compared to the intestinal environment. Specifically designed formulations are made by modifying the poloxamer concentration and excipients.

### Example 4

A formulation of the invention was tested for the ability to prevent bleeding using a mouse tail-snip assay. A portion of tail one centimeter in length was amputated from an anesthetized mouse, and the remaining portion of the tail was immediately submerged in 1.5 ml of test formulation. Bleeding time was recorded as time from amputation to complete cessation of bleeding. The following formulations were tested: water; a solution of 5% tranexamic acid (TXA) in water; a gelled formulation containing Poloxamer 407 (TDP); and a gelled formulation containing 5% tranexamic acid and TDP. Each formulation was tested on seven animals total over the course of three experiments.

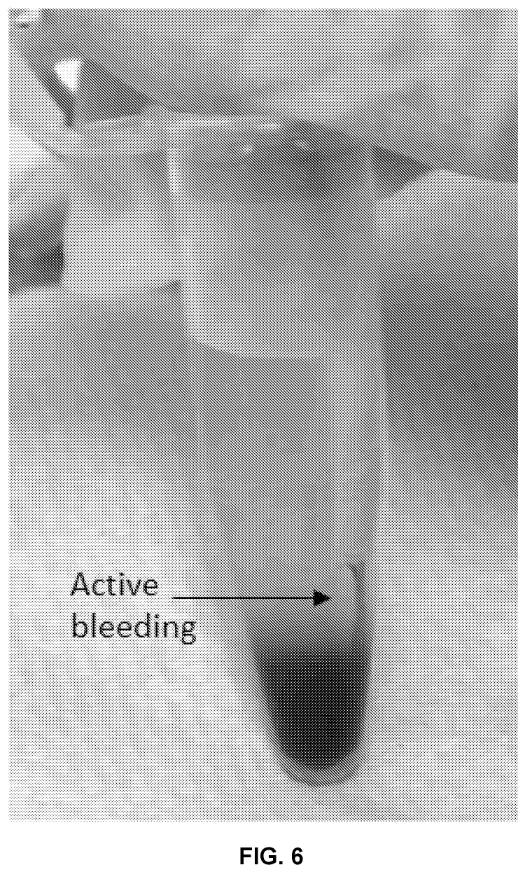

FIG. 6 is an image of an actively bleeding tail in a mouse tail-snip assay.

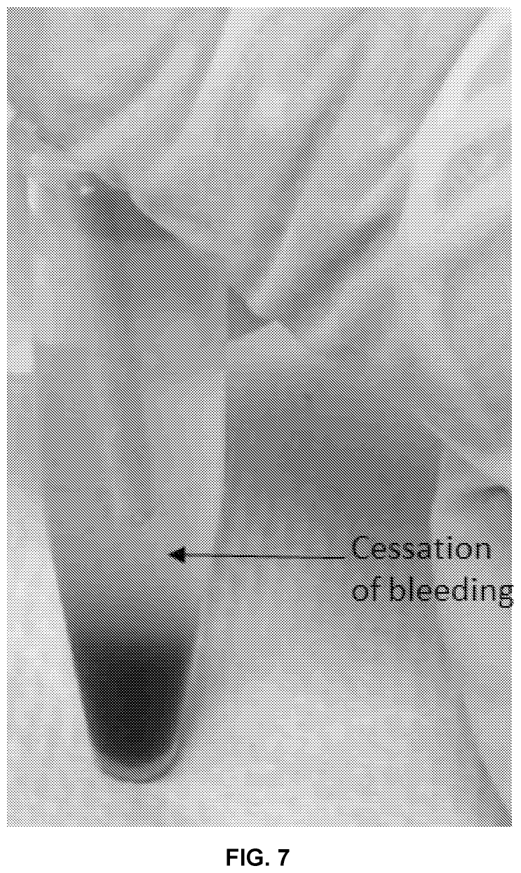

FIG. 7 is an image of a tail in which bleeding has ceased in a mouse tail-snip assay.

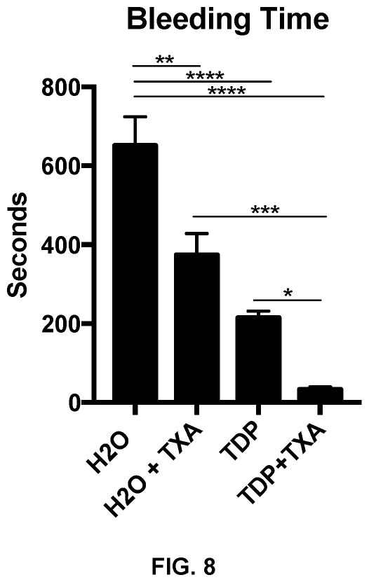

FIG. 8 is a graph showing bleeding time of mouse tails exposed to various formulations in a mouse tail-snip assay. *P<0.05, **P<0.01, ***P<0.001, ****P<0.0001, based on one-way ANOVA with the Tukey post-test.

The data show that formulations containing either TXA or TDP alone decrease bleeding time. The data further show that a formulation containing both TXA and TDP stops bleeding faster than do formulations containing either agent alone.

The results demonstrate that gelled formulations containing a polymer, such as TDP, that promotes a temperature-dependent phase transition are effective to hasten blood clotting and thus stop bleeding in mammals that have a physiological temperature above the phase-transition temperature. The results further show (1) that the aggregated form of a polymer such as TDP has inherent antihemorrhagic activity and (2) that the activity can be enhanced by the presence of another antihemorrhagic agent.

### Example 5

Formulations of the invention are tested for the ability to deliver molecular agents of different sizes. Two formulations are made by adding either tranexamic acid (TXA) or chitosan to the formulation described above in FIG. 3.

TXA is a synthetic anti-fibrinolytic drug commonly administered as a treatment for serious hemorrhage. TXA has recently been tested intravenously for the treatment of UGIB, resulting in a statistically significant reduction in the risk of death and surgical intervention.

Nevertheless, IV administration of TXA is associated with systemic side-effects and is not indicated for patients with a history of thromboembolic or ischemic events or with impaired renal functions. For general hemostatic applications, topical administration of TXA is associated with efficacy equal to or higher than IV administration, with no systemic side-effects.

Chitosan is used as a hemostatic dressing due to its proven ability to effectively control bleeding from massive hemorrhage. The hemostatic mechanism of action of chitosan remains undiscovered, but data suggest that several pathways are involved, including sorption of plasma, erythrocytes coagulation, and platelet adhesion, aggregation, and activation. The use of chitosan as a hemostatic agent in a gastrointestinal hemostatic dressing (to be used during endoscopy) has recently being proposed.

Due to the differences in properties of TXA or chitosan, analysis of formulations containing compositions containing these two agents will shed light on the utility of thermogels for delivery of a wide variety of therapeutic agents. TXA is a hydrophilic molecule of 157 Da, whereas chitosan is hydrophobic or amphoteric and has a size range of from about 10⁵-10⁶ Da. Optimized formulations for each of these agents will demonstrate the feasibility of formulations of the invention for delivery of agents, such as clotting promoters, antibiotics, prokinetics, or PPIs, that have a range of molecular properties.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A topical formulation for treatment of gastrointestinal bleeding in a subject, the formulation comprising a polymer that exists dispersed in a liquid when the formulation is below a threshold condition of one or more properties of the polymer and in an aggregated form when the formulation is above the threshold condition of the one or more properties property of the polymer, the aggregated form being effective to reduce bleeding in a gastrointestinal tract of a subject.

2. The topical formulation of claim 1, wherein the threshold condition comprises a transition temperature, and wherein the polymer exists dispersed in the liquid when the formulation is below the transition temperature and in an aggregated form when the formulation is above the transition temperature.

3. The topical formulation of claim 2, wherein the formulation exists in a liquid phase below the transition temperature and in a gel phase above the transition temperature.

4. The topical formulation of claim 2, wherein the polymer is a block copolymer comprising polyethylene glycol and polypropylene glycol.

5. The topical formulation of claim 2, wherein the transition temperature is from about 4° C. to about 38° C.

6. The topical formulation of claim 5, wherein the transition temperature is from about 32° C. to about 38° C.

7. The topical formulation of claim 1, wherein the formulation is substantially free of antihemorrhagic agents other than the polymer.

8. The topical formulation of claim 1, wherein the bleeding is in the mouth, esophagus, stomach, or small intestine.

9. The topical formulation of claim 1, wherein the bleeding is associated with a condition selected from the group consisting of caustic ingestion, Dieulafoy's lesions, duodenal ulcer, esophageal cancer, esophageal dysplasia, esophageal ulcers, esophageal varices, esophagitis, a failed endoscopy procedure, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

10. The topical formulation of claim 1, wherein the formulation comprises a consumable beverage.

11. The topical formulation of claim 1, wherein the formulation is formulated for nasogastric or endoscopic administration.

12. The topical formulation of claim 1, wherein the formulation comprises a lipid.

13. The topical formulation of claim 11, wherein the formulation comprises one selected from the group consisting of liposomes, a lipid coating, and a lipid complex.

14. A method of treating gastrointestinal bleeding in a subject, the method comprising providing locally to a gastrointestinal tract of a subject a formulation comprising a polymer that exists dispersed in a liquid when the formulation is below a threshold condition of one or more properties of the polymer and in an aggregated form when the formulation is above the threshold condition of the one or more properties of the polymer, thereby reducing bleeding in the gastrointestinal tract.

15. The method of claim 14, wherein the threshold condition comprises a transition temperature, and wherein the polymer exists dispersed in the liquid when the formulation is below the transition temperature and in an aggregated form when the formulation is above the transition temperature.

16. The method of claim 15, wherein the formulation exists in a liquid phase below the transition temperature and in a gel phase above the transition temperature.

17. The method of claim 15, wherein the polymer is a block copolymer comprising polyethylene glycol and polypropylene glycol.

18. The method of claim 15, wherein the transition temperature is from about 4° C. to about 38° C.

19. The method of claim 18, wherein the transition temperature is from about 32° C. to about 38° C.

20. The method of claim 14, wherein the formulation is provided in the liquid phase and transitions to the gel phase upon exposure to the gastrointestinal tract.

21. The method of claim 14, wherein the formulation is substantially free of antihemorrhagic agents other than the polymer.

22. The method of claim 14, wherein the bleeding is associated with a condition selected from the group consisting of caustic ingestion, Dieulafoy's lesions, duodenal ulcer, esophageal cancer, esophageal dysplasia, esophageal ulcers, esophageal varices, esophagitis, a failed endoscopy procedure, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

23. The method of claim 14, wherein the formulation is provided as a consumable beverage.

24. The method of claim 14, wherein the formulation is provided by nasogastric or endoscopic administration.

25. The method of claim 14, wherein the formulation comprises a lipid.

26. The method of claim 25, wherein the formulation comprises one selected from the group consisting of liposomes, a lipid coating, and a lipid complex.

27. A topical formulation for treatment of gastrointestinal bleeding in a subject, the formulation comprising:
a polymer that exists dispersed in a liquid when the formulation is below a threshold condition of one or more properties of the polymer and in an aggregated form when the formulation is above the threshold condition of the one or more properties of the polymer, the aggregated form being effective to reduce bleeding in a gastrointestinal tract of a subject; and
an antihemorrhagic agent that is different from the polymer.

28. The topical formulation of claim 27, wherein the threshold condition comprises a transition temperature, and wherein the polymer exists dispersed in the liquid when the formulation is below the transition temperature and in an aggregated form when the formulation is above the transition temperature.

29. The topical formulation of claim 28, wherein the formulation exists in a liquid phase below the transition temperature and in a gel phase above the transition temperature.

30. The topical formulation of claim 28, wherein the polymer is a block copolymer comprising polyethylene glycol and polypropylene glycol.

31. The topical formulation of claim 28, wherein the transition temperature is from about 4° C. to about 38° C.

32. The topical formulation of claim 31, wherein the transition temperature is from about 32° C. to about 38° C.

33. The topical formulation of claim 27, wherein the bleeding is associated with a condition selected from the group consisting of caustic ingestion, Dieulafoy's lesions, duodenal ulcer, esophageal cancer, esophageal dysplasia, esophageal ulcers, esophageal varices, esophagitis, a failed endoscopy procedure, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

34. The topical formulation of claim 27, wherein the antihemorrhagic agent is selected from the group consisting of aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, and zeolite.

35. The topical formulation of claim 27, wherein the antihemorrhagic agent is tranexamic acid.

36. The topical formulation of claim 27, wherein the formulation comprises a consumable beverage.

37. The topical formulation of claim 27, wherein the formulation is formulated for nasogastric or endoscopic administration.

38. The topical formulation of claim 27, wherein the formulation comprises a lipid.

39. The topical formulation of claim 38, wherein the formulation comprises one selected from the group consisting of liposomes, a lipid coating, and a lipid complex.

40. A method of treating gastrointestinal bleeding in a subject, the method comprising providing locally to a gastrointestinal tract of a subject a formulation comprising: a polymer that exists dispersed in a liquid when the formulation is below a threshold condition of one or more properties of the polymer and in an aggregated form when the formulation is above the threshold condition of the one or more properties of the polymer, and an antihemorrhagic agent that is different from the polymer, thereby reducing bleeding in the gastrointestinal tract.

41. The method of claim 40, wherein the threshold condition comprises a transition temperature, and wherein the polymer exists dispersed in the liquid when the formulation is below the transition temperature and in an aggregated form when the formulation is above the transition temperature.

42. The method of claim 41, wherein the formulation exists in a liquid phase below the transition temperature and in a gel phase above the transition temperature.

43. The method of claim 41, wherein the polymer is a block copolymer comprising polyethylene glycol and polypropylene glycol.

44. The method of claim 41, wherein the transition temperature is from about 4° C. to about 38° C.

45. The method of claim 44, wherein the transition temperature is from about 32° C. to about 38° C.

46. The method of claim 40, wherein the formulation is provided in the liquid phase and transitions to the gel phase upon exposure to the gastrointestinal tract.

47. The method of claim 40, wherein the antihemorrhagic agent is selected from the group consisting of aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, and zeolite.

48. The method of claim 47, wherein the antihemorrhagic agent is tranexamic acid.

49. The method of claim 40, wherein the bleeding is associated with a condition selected from the group consisting of caustic ingestion, Dieulafoy's lesions, duodenal ulcer, esophageal cancer, esophageal dysplasia, esophageal ulcers, esophageal varices, esophagitis, a failed endoscopy procedure, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

50. The method of claim 40, wherein the formulation is provided as a consumable beverage.

51. The method of claim 40, wherein the formulation comprises a lipid.

52. The method of claim 51, wherein the formulation comprises one selected from the group consisting of liposomes, a lipid coating, and a lipid complex.

53. A method of preventing a recurrence of gastrointestinal bleeding in a subject that has previously had gastrointestinal bleeding, the method comprising: providing locally to a gastrointestinal tract of a subject that has previously had gastrointestinal bleeding and that presently does not have gastrointestinal bleeding, a formulation comprising a polymer that exists dispersed in a liquid when the formulation is below a threshold condition of one or more properties of the polymer and in an aggregated form when the formulation is above the threshold condition of the one or more properties of the polymer, thereby preventing a recurrence of bleeding in the gastrointestinal tract.

54. The method of claim 53, wherein the threshold condition comprises a transition temperature, and wherein the polymer exists dispersed in the liquid when the formulation is below the transition temperature and in an aggregated form when the formulation is above the transition temperature.

55. The method of claim 54, wherein the formulation exists in a liquid phase below the transition temperature and in a gel phase above the transition temperature.

56. The method of claim 54, wherein the polymer is a block copolymer comprising polyethylene glycol and polypropylene glycol.

57. The method of claim 54, wherein the transition temperature is from about 4° C. to about 38° C.

58. The method of claim 57, wherein the transition temperature is from about 32° C. to about 38° C.

59. The method of claim 53, wherein the formulation is provided in the liquid phase and transitions to the gel phase upon exposure to the gastrointestinal tract.

60. The method of claim 53, wherein the formulation is substantially free of antihemorrhagic agents other than the polymer.

61. The method of claim 53, wherein the previous gastrointestinal bleeding is associated with a condition selected from the group consisting of caustic ingestion, Dieulafoy's lesions, duodenal ulcer, esophageal cancer, esophageal dysplasia, esophageal ulcers, esophageal varices, esophagitis, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

62. The method of claim 53, wherein the formulation is provided as a consumable beverage.

63. The method of claim 53, wherein the formulation is provided by nasogastric or endoscopic administration.

64. The method of claim 53, wherein the formulation comprises a lipid.

65. The method of claim 64, wherein the formulation comprises one selected from the group consisting of liposomes, a lipid coating, and a lipid complex.

66. A method of preventing a recurrence of gastrointestinal bleeding in a subject that has previously had gastrointestinal bleeding, the method comprising:
providing locally to a gastrointestinal tract of a subject that has previously had gastrointestinal bleeding and that presently does not have gastrointestinal bleeding, a formulation comprising: a polymer that exists dispersed in a liquid when the formulation is below a threshold condition of one or more properties of the polymer and in an aggregated form when the formulation is above the threshold condition of the one or more properties of the polymer; and an antihemorrhagic agent that is different from the polymer, thereby preventing a recurrence of bleeding in the gastrointestinal tract.

67. The method of claim 66, wherein the threshold condition comprises a transition temperature, and wherein the polymer exists dispersed in the liquid when the formulation is below the transition temperature and in an aggregated form when the formulation is above the transition temperature.

68. The method of claim 67, wherein the formulation exists in a liquid phase below the transition temperature and in a gel phase above the transition temperature.

69. The method of claim 67, wherein the polymer is a block copolymer comprising polyethylene glycol and polypropylene glycol.

70. The method of claim 67, wherein the transition temperature is from about 4° C. to about

71. The method of claim 70, wherein the transition temperature is from about 32° C. to about 38° C.

72. The method of claim 66, wherein the formulation is provided in the liquid phase and transitions to the gel phase upon exposure to the gastrointestinal tract.

73. The method of claim 66, wherein the antihemorrhagic agent is selected from the group consisting of aminocaproic acid, antihemophiliac factor, anti-inhibitor coagulant complex (heat-treated), aprotinin, avatrombopag, carbazochrome, chitosan, collagen, emicizumab, enbucrilate, factor IX, feracrylum, fibrinogen, fostamatinib, gelatin, goserelin, kaolin, lusutrombopag, n-butyl 2-cyanoacrylate, oprelvekin, thrombin, tranexamic acid, vitamin K, and zeolite.

74. The method of claim 73, wherein the antihemorrhagic agent is tranexamic acid.

75. The method of claim 66, wherein the previous gastrointestinal bleeding is associated with a condition selected from the group consisting of caustic ingestion, Dieulafoy's lesions, duodenal ulcer, esophageal cancer, esophageal dysplasia, esophageal ulcers, esophageal varices, esophagitis, foreign body ingestion, gastric antral vascular ectasia, gastric cancer, gastric dysplasia, gastric ulcer, gastric varices, gastritis, hematobilia, hemosuccus pancreaticus, iatrogenic bleeding, Mallory-Weiss tear, severe superior mesenteric artery syndrome, and vascular malformation.

76. The method of claim 66, wherein the formulation is provided as a consumable beverage.

77. The method of claim 66, wherein the formulation is provided by nasogastric or endoscopic administration.

78. The method of claim 66, wherein the formulation comprises a lipid.

79. The method of claim 78, wherein the formulation comprises one selected from the group consisting of liposomes, a lipid coating, and a lipid complex.
